(19) United States
(12) Patent Application Publication
(10) Pub. No.: US 2020/0222727 A1
(43) Pub. Date: Jul. 16, 2020

(54) Therapeutic Applicator Positioning System with Passive and Active Positioning

(71) Applicant: Profound Medical Inc., Mississauga (CA)

(72) Inventors: Mark de Bruyn, Mississauga (CA); Ting Wong, Mississauga (CA); Benny Yeung, Mississauga (CA)

(21) Appl. No.: 16/248,246

(22) Filed: Jan. 15, 2019

(51) Int. Cl. A61N 7/02; A61B 8/00

(52) U.S. Cl. CPC A61N 7/02; A61N 2007/0052; A61B 8/4245; A61B 8/4483

(57) ABSTRACT

A therapeutic applicator is positioned using a positioning system that includes a passive positioning assembly and an active positioning assembly. Each assembly has multiple degrees of freedom. The passive positioning assembly sets, with respect to a base of the system, an initial forward-backward position of the active positioning assembly, an initial vertical position of the active positioning assembly, and an initial angle of tilt of the of the active positioning assembly. The active positioning system sets an axial position of the therapeutic applicator and a rotational position of the therapeutic applicator.

### TECHNICAL FIELD

This application relates generally to ultrasound therapy systems and, more particularly to reducing electromagnetic interference (EMI) generated by ultrasound positioning apparatuses.

### BACKGROUND

Ultrasonic transducers have been employed in ultrasound therapy systems to achieve therapeutic heating of diseased and other tissues. Arrays of ultrasound transducers operating to form a beam of ultrasonic energy cause a conversion of sound to thermal energy in the affected tissue areas or treatment volumes, and a subsequent beneficial rise in the temperature in the treatment volumes.

In image-guided ultrasound therapy systems, a patient and the ultrasound therapy apparatus, including an ultrasound positioning apparatus, are generally disposed in an imaging volume such as a magnetic resonance imaging (MRI) apparatus, which allows guidance of the applicator placement, and in addition allows monitoring of the treatment effect on the tissue by providing real-time data from which temperature maps can be calculated. A clinical operator can then monitor the progress of the therapy within the treatment volume or diseased tissue and manual or automated changes can be made to the ultrasound power signals based on input from the results and progress of the treatment. With proper monitoring of the heating effect, ultrasound therapy systems can be used to treat harmful cells and to controllably destroy tumors while minimizing damage to healthy tissue.

Electromagnetic interference (EMI) occurs between the MRI apparatus, the ultrasound therapy apparatus, and the ultrasound positioning apparatus. The MRI apparatus becomes more sensitive to EMI as the power of the MRI apparatus (e.g., the power of its electromagnets) decreases. For example, a 1.5 T MRI apparatus is more sensitive to EMI than a 3 T MRI apparatus.

It would be desirable to decrease EMI from the ultrasound therapy apparatus and/or the ultrasound positioning apparatus so that they can used with lower-power MRI apparatuses.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention. One or more embodiments are directed to a applicator positioning system for positioning a therapeutic applicator with respect to a target volume, comprising a passive positioning assembly operated by a plurality of manual adjustors to control a corresponding plurality of passive degrees of freedom of said passive positioning assembly; and an active positioning assembly operated by a plurality of rotary electric motors to control a corresponding plurality of active degrees of freedom of said active positioning assembly; a motor housing unit, containing said plurality of rotary electric motors; wherein the passive positioning assembly is configured to support and set the active positioning assembly into an initial position with respect to a base of said system in said plurality of passive degrees of freedom, including a first passive degree of freedom defined by a first translational adjustment unit slidably positioning the active positioning assembly at an initial forward-backward position with respect to the base, then securable in said initial forward-backward position; a second passive degree of freedom defined by a second translational adjustment unit slidably positioning the active positioning assembly at an initial vertical position with respect to the base, then securable in said initial vertical position; and a third passive degree of freedom defined by a tilt adjustment unit angularly tilting said active positioning assembly at an initial elevation angle of tilt with respect to said base, then securable at said initial elevation angle; and wherein the active positioning assembly is configured to support and set the therapeutic applicator in a plurality of active degrees of freedom using a respective plurality of rotary electric motors disposed in said motor housing unit, including a first active degree of freedom defined by an angle of rotation of said therapeutic applicator about a longitudinal axis thereof, the longitudinal axis defined by the initial elevation angle of the active positioning assembly, said first active degree of freedom controlled by a first rotary electric motor within said motor housing, the first rotary electric motor coupled to a rotating shaft that causes rotation of said therapeutic applicator about its longitudinal axis within the target volume; and a second active degree of freedom defined by axially translating the therapeutic applicator along the longitudinal axis of the applicator, said second active degree of freedom controlled by a second rotary electric motor within said motor housing, the second rotary electric motor coupled to and driving a lead screw that rotates and moves said therapeutic applicator along the longitudinal axis of the applicator for insertion and retraction of the therapeutic applicator into and out of the target volume, respectively.

Another embodiment is directed to applicator positioning system for positioning a treatment applicator with respect to a patient support platform, the positioning system comprising a first positioning assembly mountable to said support platform; a second positioning assembly mountable to the first positioning assembly; wherein the first positioning assembly is equipped with a superior-inferior translational stage that positions the system at an initial horizontal position along an axis running in a superior-inferior direction with respect to said support platform; an anterior-posterior translational stage that positions the system at an initial vertical position along an axis running in an anterior-posterior direction above said support platform; and an elevation angle tilt stage that provides an initial elevation angle of tilt to said second positioning assembly and said applicator; and wherein the second positioning assembly is equipped with a motorized and computer-controlled translational stage that positions the applicator along an axial location defined by an axis of said applicator so as to provide control of a linear insertion-and-retraction movement of said applicator along its axis, said motorized translational stage including a first rotary electric motor coupled to a lead screw rotating therewith to affect said insertion and retraction; and a motorized and computer-controlled rotational stage that positions the applicator at an angle of rotation about its axis so as to provide rotational control of said applicator, said motorized rotational stage including a second rotary electric motor coupled to a shaft rotating therewith to affect said angular control.

The motors used in the present embodiments may comprise electromagnetic radiation or radio frequency (RF) resistant motors, for example those employing some or all non-ferrous materials, e.g., ceramics, aluminum, polymers, or other materials. The motors used in the present embodiments are sometimes disposed as far as practical from a source of electromagnetic (EM) fields such as MRI imaging magnets or MRI magnet bores. In some cases, the motors are housed in EM-shielded housings including grounded Faraday cage or similar enclosures to minimize the effect of EM fields and/or RF radiation from passing through the enclosure walls between an imaging environment and the motor housing. In this way the adverse effects of the EM fields on the motors and motor controllers as well as the adverse effects of radiation from the motors on a medical imaging process are reduced or eliminated.

### BRIEF DESCRIPTION OF THE DRAWINGS

For a fuller understanding of the nature and advantages of the present concepts, reference is made to the following detailed description of preferred embodiments and in connection with the accompanying drawings, in which:

FIG. 1 is a diagram of one type of system in which at least some of the apparatus and/or methods disclosed herein are employed, in accordance with at least some embodiments;

FIG. 2 illustrates an example of an ultrasound applicator that can be used in system illustrated in FIG. 1;

FIG. 3 is a perspective view of a positioning apparatus for a therapeutic applicator according to one or more embodiments;

FIG. 4 is an enlarged view of a portion of the positioning apparatus illustrated in FIG. 3;

FIG. 5 illustrates the positioning apparatus at a middle axial position, a middle vertical position, and a downward angular position;

FIG. 6 is an elevated perspective view of a therapeutic applicator support body according to one or more embodiments;

FIG. 7 is a lower perspective view of the first and second clamps illustrated in FIG. 6;

FIG. 8 is a cross section of the therapeutic applicator support body through plane 8-8 in FIG. 6;

FIG. 9 illustrates an example of a gear in a handle of a therapeutic applicator that can engage with the first gear mechanism in the therapeutic applicator support body;

FIG. 10 is a block diagram of therapeutic applicator support body according to one or more embodiments;

FIGS. 11, 12, and 13 illustrate a therapeutic applicator disposed over therapeutic applicator support body according to one or more embodiments;

FIG. 14 is a flow chart 1400 of a method for positioning a therapeutic applicator with a positioning system that includes a passive positioning assembly and an active positioning assembly;

FIG. 15 is a flow chart of a method for indexing a rotational position of a therapeutic applicator; and

FIG. 16 is a flow chart of a method for determining a presence of a therapeutic applicator.

### DETAILED DESCRIPTION

A positioning system for a therapeutic applicator includes a passive positioning apparatus and an active positioning apparatus. The passive positioning apparatus manually sets and locks an initial position of the active positioning apparatus, with respect to a base of the system, using a plurality of passive degrees of freedom. The passive degrees of freedom include an initial forward-back position of the active positioning assembly, an initial vertical position of the active positioning assembly, and an initial angle of tilt of the active positioning assembly. The active positioning apparatus includes processor-controller motors to adjust the position of the therapeutic applicator using a plurality of active degrees of freedom. The active degrees of freedom include an insertion-retraction position of the therapeutic applicator (e.g., the tip of the therapeutic applicator) with respect to an imaging volume and a rotational position or orientation of the therapeutic applicator with respect to its longitudinal axis. The rotational position or orientation of the therapeutic applicator corresponds to the rotational position or orientation the therapeutic applicator's tip and thus the angular direction that energy is emitted from the tip.

The active positioning system can include a rotational indexing apparatus to determine a home position or reference rotational position of the therapeutic applicator. The rotational indexing apparatus includes an optical port disposed at a distal end of the active positioning apparatus and a light source/detector disposed at a proximal end of the active positioning apparatus. The optical port and light source/detector are optically coupled to an optical fiber that extends therebetween. The optical port outputs light generated by the light source/detector. The optical source/detector can be implemented as separate optical energy source and optical energy detector elements (e.g., diodes, LEDs, analog, digital, or other equivalent devices), or the source and detector elements may be implemented as a combined or monolithic architecture that can separately or simultaneously send and/or receive optical energy as would be appropriate for a given application and appreciated by one of skill in the art. When the therapeutic applicator is in the home or reference rotational position, at least a portion of light is reflected back into the optical port where it is detected by the light source/detector. When the therapeutic applicator is not in the home or reference rotational position, no light (or substantially no light) is reflected back into the optical port.

The active positioning system can also include an applicator presence detection apparatus. The applicator presence apparatus includes a second optical port disposed at a distal end of the active positioning apparatus and a light detector disposed at a proximal end of the active positioning apparatus. The optical port and light source/detector are optically coupled to an optical fiber that extends therebetween. When a therapeutic applicator is disposed or mounted on the active positioning system, the second optical port is blocked and no or minimal light (e.g., below a threshold intensity) enters the second optical port. When a therapeutic applicator is disposed or mounted on the active positioning system, the second optical port is not blocked and ambient light (e.g., at or above the threshold intensity) enters the second optical port.

The electrical and electromechanical components of the active positioning apparatus are disposed in an electromagnetic shielding chamber within the active positioning apparatus at a distal end thereof. The electromagnetic shielding chamber reduces EMI between the active positioning apparatus and the environment in which it operates, which can include an MR apparatus.

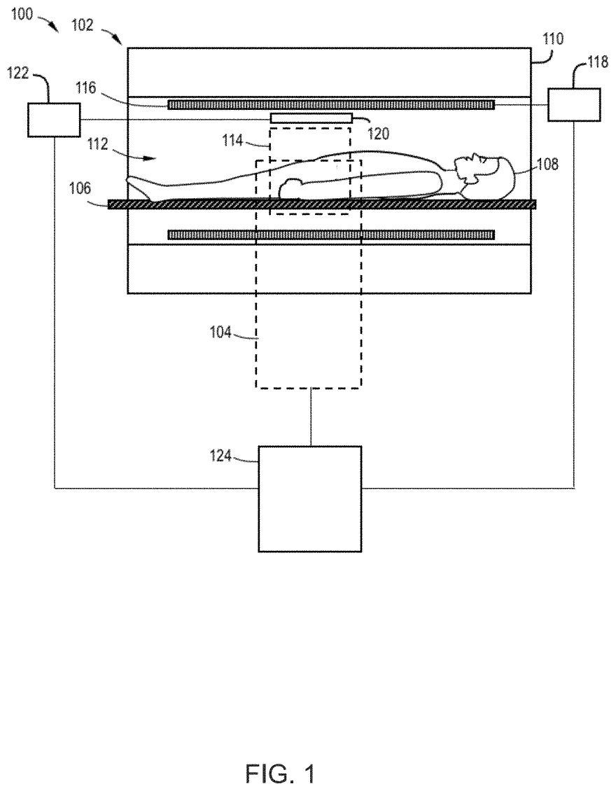

FIG. 1 is a diagram of one type of medical system 100 in which at least some of the apparatus, systems, and/or methods disclosed herein are employed, in accordance with at least some embodiments. The system 100 includes a patient support 106 (on which a patient 108 is shown), a magnetic resonance system 102 and an image guided energy delivery system 104.

The magnetic resonance system 102 includes a magnet 110 disposed about an opening 112, an imaging zone 114 in which the magnetic field is strong and uniform enough to perform magnetic resonance imaging, a set of magnetic field gradient coils 116 to change the magnetic field rapidly to enable the spatial coding of MRI signals, a magnetic field gradient coil power supply 118 that supplies current to the magnetic field gradient coils 116 and is controlled as a function of time, a transmit/receive coil 120 (also known as a “body” coil) to manipulate the orientations of magnetic spins within the imaging zone 114, a radio frequency transceiver 122 connected to the transmit/receive coil 120, and a computer 124, which performs tasks (by executing instructions and/or otherwise) to facilitate operation of the MRI system 102 and is coupled to the radio frequency transceiver 122, the magnetic field gradient coil power supply 118, and the image guided energy delivery system 104.

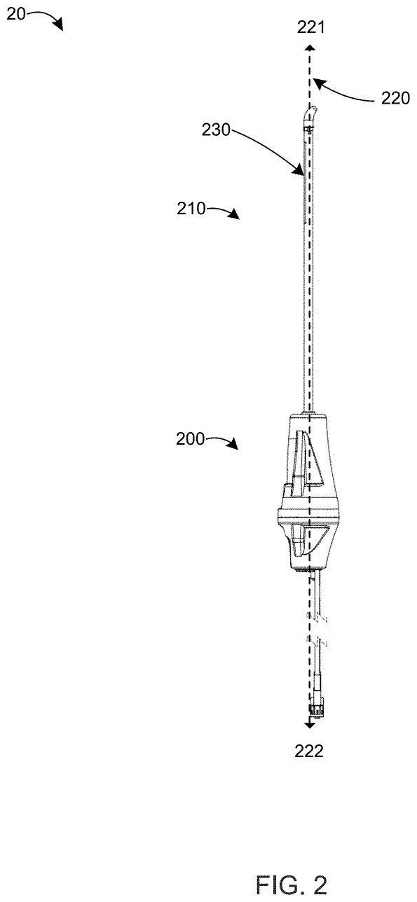

The image guided energy delivery system 104 includes a therapeutic applicator, such as an ultrasound applicator, to perform image guided therapy (e.g., thermal therapy) in multiple angular directions to treat a treatment region. An example of a therapeutic applicator that can be used in system 100 is ultrasound applicator 20 illustrated in FIG. 2. The ultrasound applicator 20 can be mounted on a positioning apparatus, embodiments of which are described herein.

The MRI computer 124 can include more than one computer in some embodiments, which can be dedicated for the MRI system 102. In at least some embodiments, the MRI computer 124 and/or one or more other computing devices (not shown) in and/or coupled to the system 100 may also perform one or more tasks (by executing instructions and/or otherwise) to implement one or more aspects and/or embodiments disclosed herein (or portion(s) thereof) to control the rotational position and insertion-retraction position of the therapeutic applicator, for example with respect to a target volume.

One or more of the computers, including computer 124, can include a treatment plan for the patient 108 that includes the target treatment region and the desired or minimal energy (e.g., thermal) dose for the target treatment region. The computer(s) can use images from the magnetic resonance system 102 to image guide the rotational position and insertion-retraction position of the therapeutic applicator. In some embodiments, one or more dedicated computers control the image guided energy delivery system 104. Some or all of the foregoing computers can be in communication with one another (e.g., over a local area network, a wide area network, a cellular network, a WiFi network, or other network), for example through a software-controlled link to a communication network.

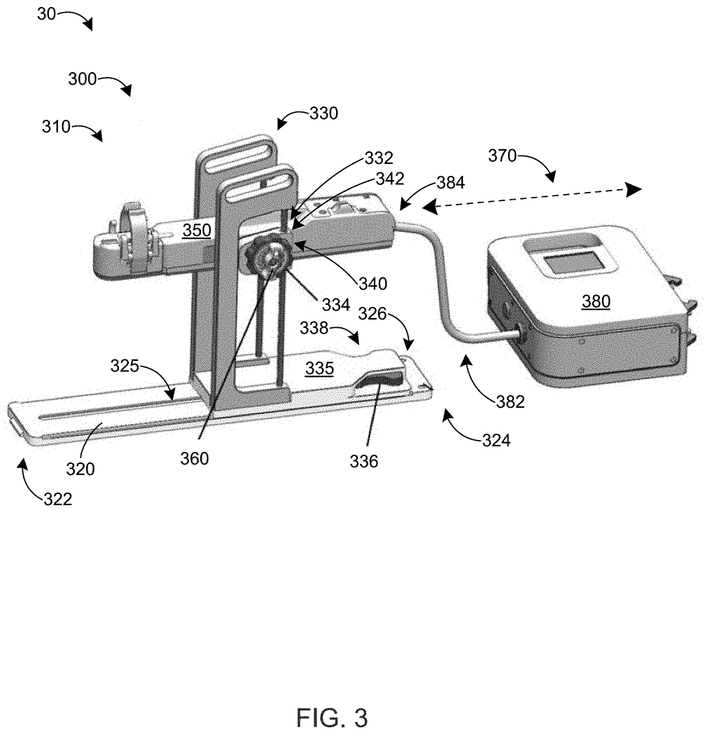

FIG. 3 is a perspective view of a positioning apparatus 30 for a therapeutic applicator according to one or more embodiments. The positioning apparatus 30 includes a passive positioning assembly 300 and an active positioning assembly 310. The passive positioning assembly 300 and the active positioning assembly 310 each provides multiple degrees of freedom. The passive positioning assembly 300 has three degrees of freedom for setting an initial position of the active positioning system 310 and a therapeutic applicator disposed or mounted thereon. The active positioning system 310 has two degrees of freedom for positioning the therapeutic applicator, for example with respect to a target volume.

The passive positioning assembly 300 includes a base 320 and support arms 330. The support arms 330 are mechanically coupled to and extend from a support base 335, which can slidably engage the base 320 along groove 325. The support base 335 includes a mechanical lock button 336 on a distal end 338 of the support base 335 to releasably lock the axial position of the support base 335 along the groove 325. When the mechanical lock button 336 is pushed in, the lock is released and the support base 335 can move along the groove 325. When the mechanical lock button 336 is released, the lock is set and the position of the support base 335 along the groove 325 is fixed. One or more screws 326 can be driven through a respective hole(s) in the base 320 and into a lower surface (e.g., a table) to secure the position and orientation of the base 320 with respect to the lower surface, which can prevent the base 320 from moving while the positioning apparatus 30 is adjusted.

In operation, a user can adjust the axial position of the therapeutic applicator support body 350 by (a) pressing in the mechanical lock button 336 to release the lock, (b) moving the support base 335 axially towards the proximal end 322 or distal end 324 of the base 320 along axis 370 (i.e., closer to or away from a patient located near the proximal end 322 of the base 320) until the therapeutic applicator support body 350 is located at a desired axial position (e.g., with respect to a target location in or on a patient) or forward-backward position, and (c) releasing the mechanical lock button 336 to lock the axial position of the support base 335 and therapeutic applicator support body 350.

Thus, the passive positioning assembly 300 provides a first degree of freedom for linearly or axially configuring or positioning the active positioning assembly 310, which includes the therapeutic applicator support body 350, along axis 370 (e.g., a horizontal axis) in a forward-backward position with respect to base 320. Since a therapeutic applicator can be disposed on the therapeutic applicator support body 350, the passive positioning assembly 300 also provides a first degree of freedom for linearly or axially positioning the therapeutic applicator along axis 370. The linear or axial configuration or positioning provided by the passive positioning assembly 300 sets the forward-backward or proximal-distal position of the active positioning assembly 310 including therapeutic applicator support body 350 and a therapeutic applicator disposed thereon, and the linear or axial distance from (a) the therapeutic applicator support body 350, and a therapeutic applicator disposed thereon, to (b) a target location in, on, or proximal to a patient.

Each support arm 330 includes a rod 332 that extends vertically from the support base 335. Each rod 332 extends through a hole 342 in a respective support body 340 to allow the support body 340 to slide upwards and downwards along the respective rod 332. A respective vertical positioning knob 334 can be tightened to fix the position of each support body 340 with respect to the corresponding rod 332. The support bodies 340 are mechanically coupled to therapeutic applicator support body 350. The vertical position of the therapeutic applicator support body 350 can be adjusted by sliding the support bodies 340 upwards and downwards along the rods 332.

In operation, a user can adjust the vertical position of the therapeutic applicator support body 350 by (a) loosening the vertical positioning knob 334, (b) sliding the support bodies 340 along the rods 332 until the therapeutic applicator support body 350 is located at a desired vertical position (e.g., with respect to a target location in or on a patient), and (c) tightening the vertical positioning knob 334.

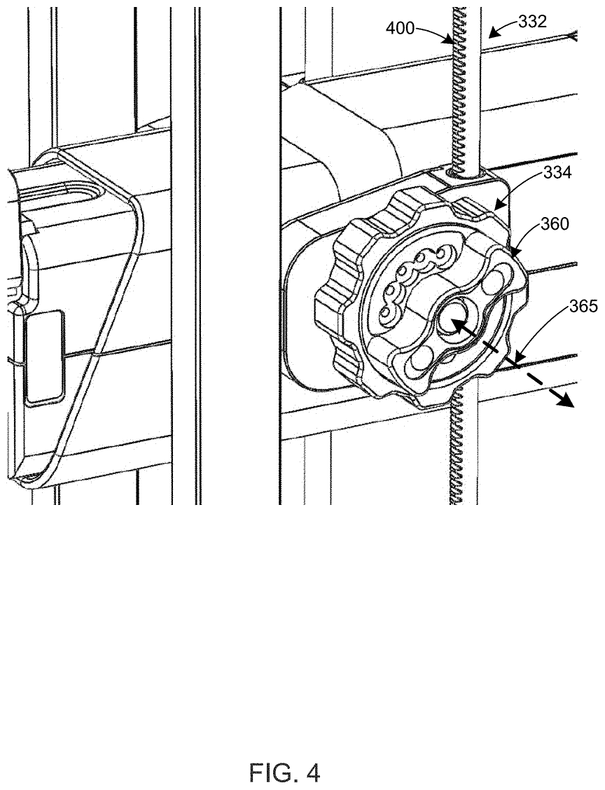

In some embodiments, the vertical positioning knob 334 is mechanically coupled to a gear that mechanically engages teeth 400 on the respective rod(s) 332 (e.g., as a rack and pinion connection), as illustrated in FIG. 4. Rotating the vertical positioning knob(s) 334 causes the gear to rotate with respect to the teeth 400 to cause the gear and support bodies 340 to move axially (i.e., vertically) along the length of the rods 332.

Thus, the passive positioning assembly 300 provides a second degree of freedom for linearly or axially configuring or positioning the active positioning assembly 310, which includes the therapeutic applicator support body 350, along a vertical axis. Since a therapeutic applicator can be disposed on the therapeutic applicator support body 350, the passive positioning assembly 300 also provides a second degree of freedom for linearly or axially positioning the therapeutic applicator along the vertical axis. The linear or axial configuration or positioning provided by the passive positioning assembly 300 sets the vertical or upward-downward position of the therapeutic applicator support body 350, and a therapeutic applicator disposed thereon, which can align the therapeutic applicator with a target location in or on a patient.

The passive positioning assembly 300 also includes an angular positioning knob 360 that is mechanically coupled to the support bodies 340 and to the therapeutic applicator support body 350. The angular positioning knob 360 can be loosened (i.e., rotated in a first direction) to allow the therapeutic applicator support body 350 to pivot or rotate with respect to a rotation axis 365, as illustrated in FIG. 4. In some embodiments, a shaft extends along the rotation axis 365 through a hole defined through at least a portion of the therapeutic applicator support body 350, and the therapeutic applicator support body 350 pivots or rotates with respect to the shaft. When the therapeutic applicator support body 350 is rotated to a desired angular position (e.g., with respect to a target location in, on, or proximal to a patient), the angular positioning knob 360 can be tightened (i.e., rotated in a second direction) to lock the angular position of the therapeutic applicator support body 350. In some embodiments, tightening the angular positioning knob 360 also locks the vertical position of the support bodies 340 along the rods 332, in which case the vertical positioning knob 334 optionally does not lock the vertical position of the support bodies 340.

In operation, a user can adjust the angular or rotational position of the therapeutic applicator support body 350 by (a) loosening the angular positioning knob 360, (b) rotating the therapeutic applicator support body 350 with respect to the rotation axis 365 until the therapeutic applicator support body 350 is located at a desired angular position (e.g., with respect to a target location in, on, or proximal to a patient), and (c) tightening the angular positioning knob 360.

Thus, the passive positioning assembly 300 provides a third degree of freedom for configuring or setting the angular position or orientation of the active positioning assembly 310, which includes the therapeutic applicator support body 350. Since a therapeutic applicator can be disposed on the therapeutic applicator support body 350, the passive positioning assembly 300 also provides a third degree of freedom for configuring or setting the angular position or orientation of the therapeutic applicator, and thus the angular direction of energy emitted at the tip of the therapeutic applicator into the target volume. The angular position/orientation configuration or setting provided by the passive positioning assembly 300 sets the angular position or orientation of the therapeutic applicator support body 350, and a therapeutic applicator disposed thereon, which can align the therapeutic applicator with the target location in or on a patient.

In FIG. 3, the therapeutic applicator support body 350 is positioned such that it is at a distal axial position, a high vertical position, and a neutral angular position. It is noted that the angular position of the therapeutic applicator support body 350 can be determined by the angle between an axis that is parallel to the surface or length of the therapeutic applicator support body 350 (e.g., axis 370 in FIG. 3) and a reference axis. In some embodiments, the reference axis is parallel to the length of the base 320 (e.g., parallel to groove 325) and orthogonal to the support arms 330. When the reference axis is parallel to the length of the base 320, the angular position of the therapeutic applicator support body 350 is 0° in FIG. 3.

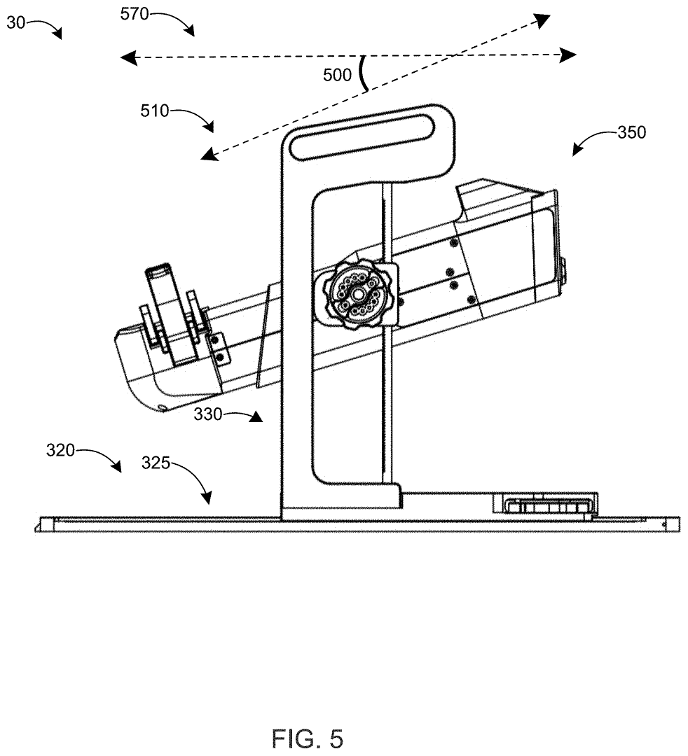

FIG. 5 illustrates the positioning apparatus 30 at a middle axial position, a middle vertical position, and a downward angular position. As discussed above, the angular position of the therapeutic applicator support body 350 can be determined by angle 500 defined between an axis 510 that is parallel to the length of the applicator support body 350 a reference axis, such as reference axis 570. Reference axis 570 is parallel to the length of the base 320 (e.g., parallel to groove 325 or z-axis) and orthogonal to the support arms 330 (or y-axis). Thus, angle 500 is determined with respect to a plane (e.g., the y-z plane) defined by axis 510 and reference axis 570, the plane being orthogonal to a width of the base 320.

The active positioning assembly 310 includes the therapeutic applicator support body 350 and a processor-based motor controller 380, which are electrically coupled to each other by a cable 382. The motor controller 380 can send control signals to one or more electromechanical positioners disposed in the therapeutic applicator support body 350 via a motor controller interface 384 in the therapeutic applicator support body 350. The control signals (e.g., axial-position commands and rotational-position commands) can cause the state of the electromechanical positioner(s) to change, for example to cause the therapeutic applicator to translate and/or rotate. The motor controller 380 can receive input data from another computer (e.g., computer 124) for image-guided control and feedback of the active positioning assembly 310.

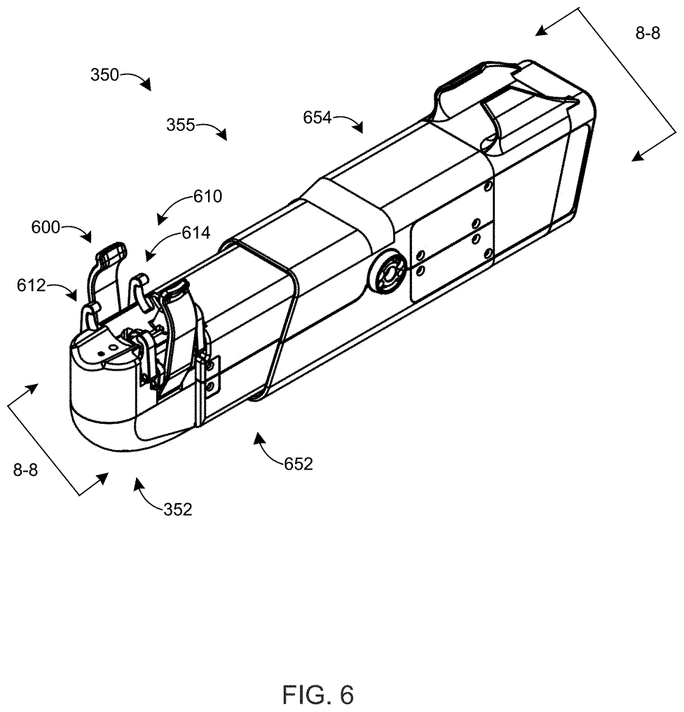

FIG. 6 is an elevated perspective view of the therapeutic applicator support body 350 according to one or more embodiments. The therapeutic applicator support body 350 includes one or more exposed surfaces 355 on which a therapeutic applicator can be disposed. First and second clamps 600, 610 are disposed at the distal end 352 of the therapeutic applicator support body 350. The first and second clamps 600, 610 are mechanically coupled to each other, and the second clamp 610 is configured to operate mechanically according to the position of the first clamp 600. For example, opening the first clamp 600 causes the second clamps 610 to open and closing the first clamp 600 causes the second clamps 610 to close. The first clamp 600 is configured to be operated with a user's hand such as by pressing inwardly or outwardly on the first clamp 600 with the user's fingers. The second clamps 610 include a distal clamp 612 and a proximal clamp 614. The distal clamp 612 is located on the distal side of the first clamp 600 proximal to distal end 352, and the proximal clamp 614 is located on the proximal side of the first clamp 600.

With the clamps 600, 610 in the open position, a therapeutic applicator can be placed on the exposed surface 355 of the therapeutic applicator support body 350 such that the therapeutic applicator handle (e.g., therapeutic applicator handle 200 illustrated in FIG. 2) is disposed proximal to the second clamp 610. The therapeutic applicator is placed on the therapeutic applicator support body 350 such that the tip of the therapeutic applicator (e.g., tip 210 illustrated in FIG. 2) is disposed distally from its handle and the clamps 600, 610. In the closed position, the second clamp 610 applies an inward force on the therapeutic applicator handle to secure the position and orientation of the therapeutic applicator. The proximal and distal clamps 612, 614 provide two points of contact with the therapeutic applicator handle to further secure the position and orientation of the therapeutic applicator.

FIG. 6 also illustrates that the therapeutic applicator support body 350 includes first and second portions 652, 654. The first portion 652 can slide towards or away from the second portion 654 according to the position of an electromechanical positioner disposed in the therapeutic applicator support body 350. The second clamp 610 mechanically couples the therapeutic applicator to the first portion 652 of the therapeutic applicator support body 350 such that the therapeutic applicator moves linearly or axially with the first portion 652 of the therapeutic applicator support body 350.

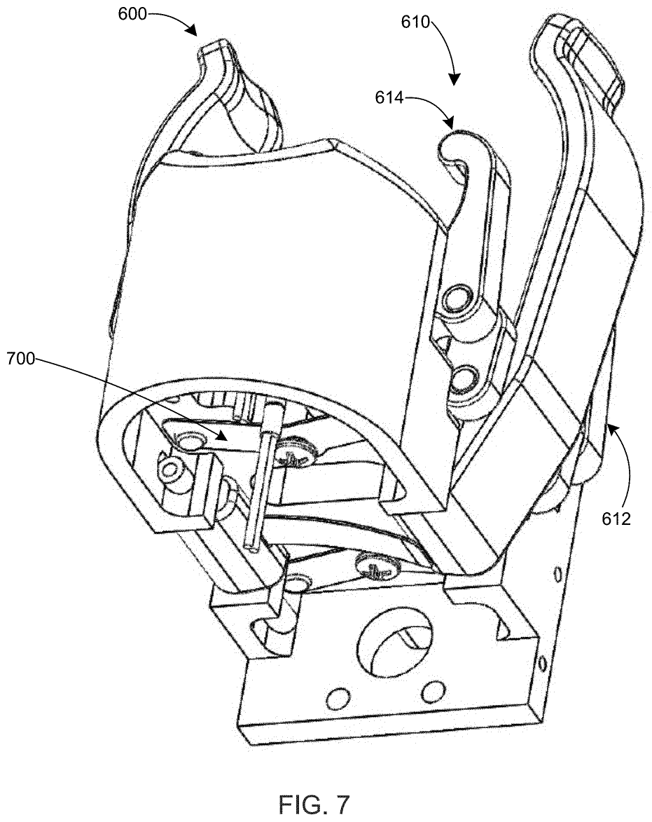

FIG. 7 is a lower perspective view of the first and second clamps 600, 610. The proximal clamps 612 are mechanically mounted to the first clamp 600. Though not illustrated in FIG. 7, the distal clamps 614 are also mechanically mounted to the first clamp 600. The first clamp 600 is mechanically coupled to a pair of rotatable arms 700 that can open and close by pivoting on respective pivot points, or, like scissors. The rotatable arms 700 can provide an inward force on the proximal and distal clamps 612, 614 to further secure them to the therapeutic applicator handle. The rotatable arms 700 can also provide an outward force on the proximal and distal clamps 612, 614 to open them from the closed position.

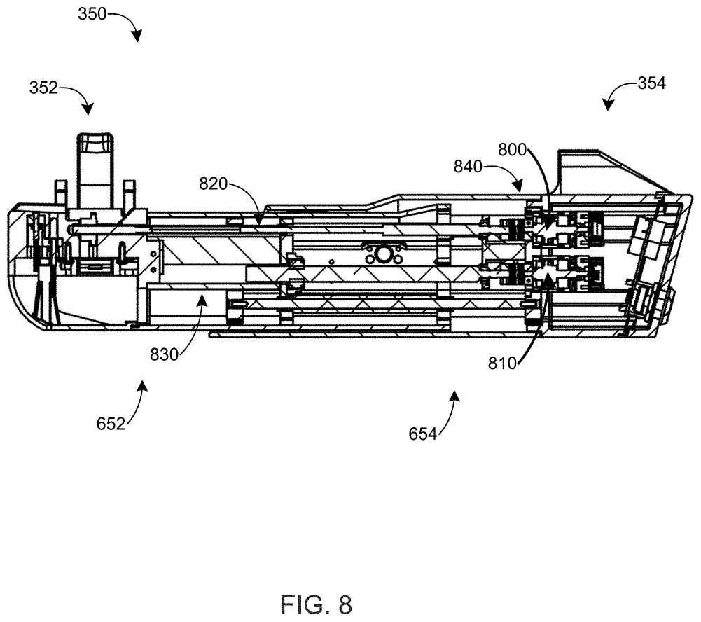

FIG. 8 is a cross section of therapeutic applicator support body 350 through plane 8-8 in FIG. 6. The cross section reveals first and second motors 800, 810 disposed at the proximal end 354 of the therapeutic applicator support body 350. The motors 800, 810 are mechanically coupled to first and second lead screws 820, 830, respectively, that extend to the distal end 352 of the therapeutic applicator support body 350. The first and second motors 800, 810 and the first and second lead screws 820, 830 are part of the active positioning assembly 310, discussed above.

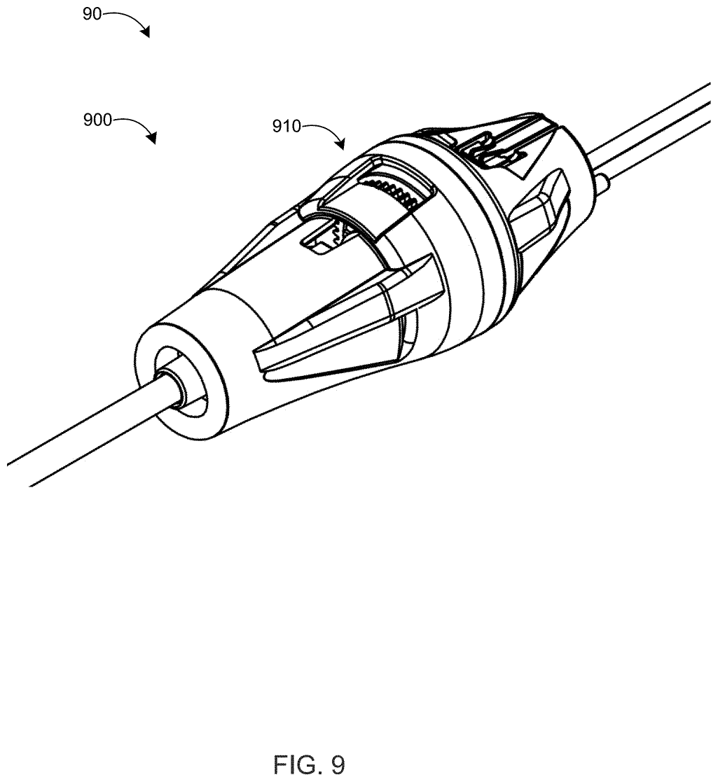

At the distal end 352, the first lead screw 820 is coupled to a first gear mechanism that causes the therapeutic applicator to rotate about its longitudinal axis. Specifically, the first gear mechanism is coupled to a corresponding gear that extends about the circumference of the therapeutic applicator. The first motor 800 causes the first lead screw 820 to rotate, which causes the first gear mechanism to rotate thereby rotating the therapeutic applicator. FIG. 9 illustrates an example of a gear 910 in a handle 900 of a therapeutic applicator 90 that can engage with the first gear mechanism in the therapeutic applicator support body.

Thus, the active positioning assembly 310, including first motor 800 and first lead screw 820, provides a fourth degree of freedom for configuring or setting the rotational or azimuthal position or orientation of the therapeutic applicator with respect to a reference position or orientation of the therapeutic applicator. For example, the fourth degree of freedom can be used to rotate the therapeutic applicator such that energy (e.g., ultrasound) emitted from the therapeutic applicator's tip is directed to or towards a target volume according to a treatment plan.

In addition, the second lead screw 830 is coupled to a second gear mechanism, at the distal end 352, that causes the first portion 652 of the therapeutic applicator support body 350 to slide towards or away from the second portion 654 of the therapeutic applicator support body 350, depending on the direction of rotation of the second lead screw 830 and second motor 810.

Thus, the active positioning assembly 310 provides a fifth degree of freedom for further configuring or setting the distal-proximal, axial, or forward-backward position of the therapeutic applicator along its axial position or orientation (as configured or set by the third degree of freedom). For example, the fifth degree of freedom can further insert or retract the therapeutic applicator into or out of a target volume or location along the longitudinal axis of the therapeutic applicator, which is determined by its axial position or orientation. The distal-proximal position of the therapeutic applicator determines the corresponding position of the therapeutic applicator's tip that emits energy, such as ultrasound, towards a target volume. So adjusting the distal-proximal position of the therapeutic applicator adjusts the axial position at which energy is emitted the therapeutic applicator tip such that the energy is directed towards a target volume according to a treatment plan.

The first and second motors 800, 810 are disposed in an electromagnetic interference (EMI) shielding chamber 840 that includes a grounded conducting layer such as copper, nickel, brass, and/or another grounded conducting layer. Since the positioning apparatus 30 is typically disposed near a magnetic resonance system, such as magnetic resonance system 202, during clinical use, the EMI shielding chamber 840 prevents or reduces any EMI generated by the motors 800, 810 from interfering with the environment in which the active positioning assembly 310 is operated (which can include a magnetic resonance (MR) system) and vice versa. In particular, the EMI shielding chamber 840 can be useful when operating the active positioning assembly 310 proximal to a MR system having relatively low-strength electromagnets (e.g., 1.5 T) which are more susceptible to EMI than an MR system having relatively high-strength electromagnets (e.g., 3 T).

The first and second motors 800, 810 are disposed as far away as possible from the magnetic resonance system (i.e., at the proximal end 354 of the therapeutic applicator support body 350) to further reduce EMI from the motors 800, 810 to the MR system (and vice versa). In the distal direction from the EMI shielding chamber 840, the remainder of the therapeutic applicator support body 350 does not include any electronic or electromechanical components, which is part of the design for reducing EMI.

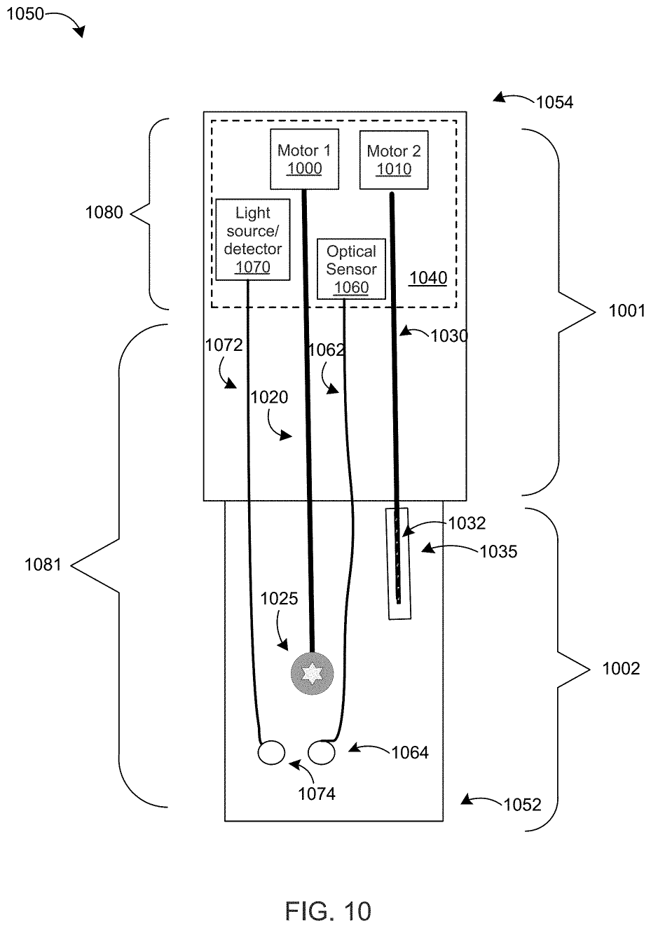

FIG. 10 is a block diagram of therapeutic applicator support body 1050 according to one or more embodiments. Therapeutic applicator support body 1050 can be the same as or different than therapeutic applicator support body 350. Therapeutic applicator support body 1050 includes first and second motors 1000, 1010 that are in mechanical communication with first and second lead screws 1020, 1030. The first and second lead screws 1020, 1030 extend from the first and second motors 1000, 1010, respectively, to the distal portion 1002 of the therapeutic applicator support body 1050. The first and second motors 1000, 1010 and the first and second lead screws 1020, 1030 can be the same as or different than first and second motors 800, 810 and the first and second lead screws 820, 830, respectively.

The first lead screw 1020 is mechanically coupled to a first geared apparatus 1025, which can be mechanically coupled to a gear (e.g., gear 900) on the handle of a therapeutic applicator. In operation, rotation by the first motor 1000 causes the first lead screw 1020 to rotate, which causes the first geared apparatus 1025 to rotate. The rotation of the first geared apparatus 1025 causes the therapeutic applicator to rotate about its longitudinal axis via a gear (e.g., gear 900) on the handle of the therapeutic applicator.

Threads on a distal portion of the second lead screw 1030 engage corresponding threads in a threaded hollow body 1035. In operation, rotation by the second motor 1010 causes the second lead screw 1030 to rotate, which causes the threads 1032 on the second lead screw 1030 to rotate with respect to the threads in the threaded hollow body 1035. The rotation of the threads 1032 on the second lead screw 1030 cause the distal portion 1002 to move away from or towards the proximal portion 1001, depending on the direction of rotation.

The therapeutic applicator support body 1050 also includes an optical sensor 1060 that is optically coupled to an optical fiber 1062 that extends to an optical port 1064 at the distal end 1052 of distal portion 1002. Any light that passes into (or is received by) the optical port 1064 passes through the optical fiber 1062 and is detected by the optical sensor 1060. The output of the optical sensor 1060 can indicate the presence or absence of light, which can correspond to the presence or absence, respectively, of a therapeutic applicator on the therapeutic applicator support body 1050. In other words, when a therapeutic applicator is disposed on the therapeutic applicator support body 1050, the optical sensor 1060 will not detect any light, or the intensity of the detected light will be below a threshold intensity level. When a therapeutic applicator is not disposed on the therapeutic applicator support body 1050, the optical sensor 1060 will detect light, or the intensity of the detected light will be at or above a threshold intensity level. Thus, the optical sensor 1060 can determine whether a therapeutic applicator is present.

The therapeutic applicator support body 1050 also includes a light source/detector 1070 that is optically coupled to an optical fiber 1072 that extends to an optical port 1074 at the distal end 1052 of distal portion 1002. The light source/detector 1070 generates light (e.g., from one or more LEDs) that passes through optical fiber 1072 and exits optical port 1074. The light can be reflected by a reflective rotational indexing body on the therapeutic applicator when the therapeutic applicator is in a first rotational position, which can correspond to a reference rotational position or a home rotational position. At least a portion of the reflected light reflected passes into the optical port 1074 and is detected by light source/detector 1070 after passing through optical fiber 1072. When the therapeutic applicator is in a rotational position other than the first rotational position, the light does not reflect from the reflective rotational indexing body. The light source/detector 1070 can generate a first output signal when the light source/detector 1070 detects reflected light and a second output signal when the light source/detector 1070 does not detect reflected light. Thus, the light source/detector 1070 can determine a reference or a home rotational position of the therapeutic applicator.

Output data signals from the optical sensor 1060 and the light source/detector 1070 are transmitted via motor controller interface 384 to controller 380. Controller 380 can

The motors 1000, 1010, the optical sensor 1060, and the light source/detector 1070 are disposed in an EMI shielding chamber 1040 at the proximal end 1054 of the proximal portion 1001 of the therapeutic applicator support body 1050. EMI shielding chamber 1040 can be the same as or different than EMI shielding chamber 840.

Therefore, the therapeutic applicator support body 1050 includes an electrical portion 1080 and a mechanical portion 1081. The electrical portion 1080 is disposed proximal to the mechanical portion 1081 so that the electrical portion 1080 is as far away from the MR system as possible to prevent or reduce any EMI between the electrical portion 1080 and the MR system. The electrical portion 1080 includes EMI shielding chamber 1040 to further reduce any such EMI. Thus, electrical portion 1080 can also be referred to as a shielded electrical portion.

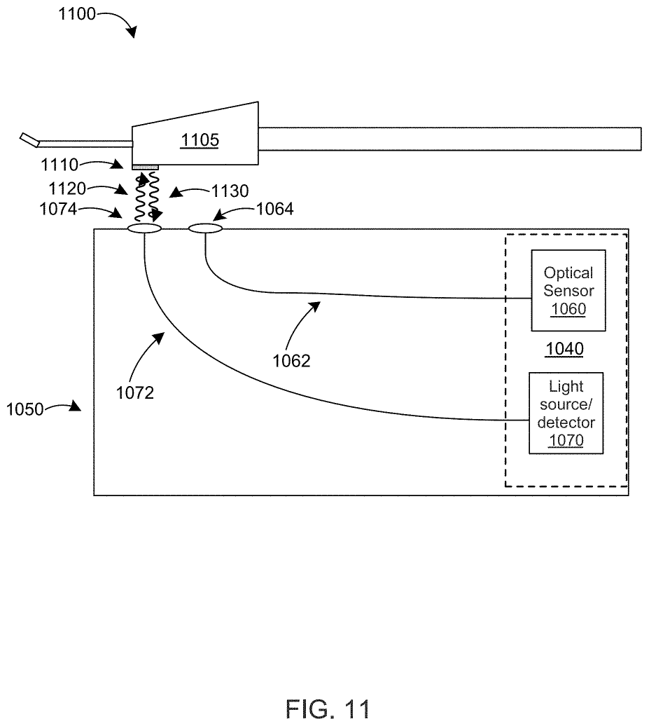

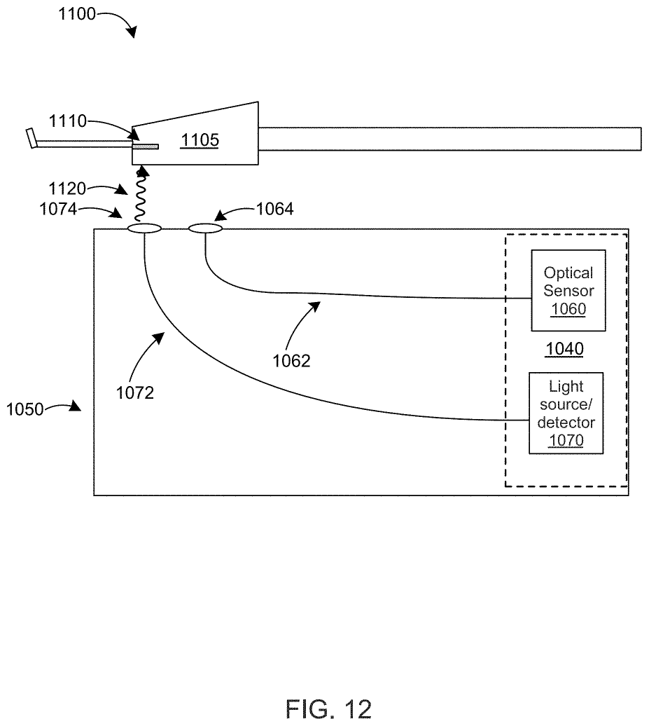

FIG. 11 illustrates a therapeutic applicator 1100 disposed over therapeutic applicator support body 1050 according to one or more embodiments. Therapeutic applicator 1100 is not illustrated as disposed on therapeutic applicator support body 1050 to illustrate the functionality of the optical sensor 1060 and light source/detector 1070. When the therapeutic applicator 1100 is in the home or reference position, reflective rotational indexing body 1110 on handle 1105 is oriented to face optical port 1074 such that light 1120 exiting or output from optical port 1074 is reflected by the reflective rotational indexing body 1110. At least a portion of the reflected light 1130 enters optical port 1074 where it is detected by light source/detector 1070 after passing through optical fiber 1072. When the therapeutic applicator 1100 is not in the home or reference position, the reflective rotational indexing body 1110 on handle 1105 is oriented away from optical port 1074 such that light 1120 exiting optical port 1074 is not reflected by the reflective rotational indexing body 1110 or handle 1105, as illustrated in FIG. 12.

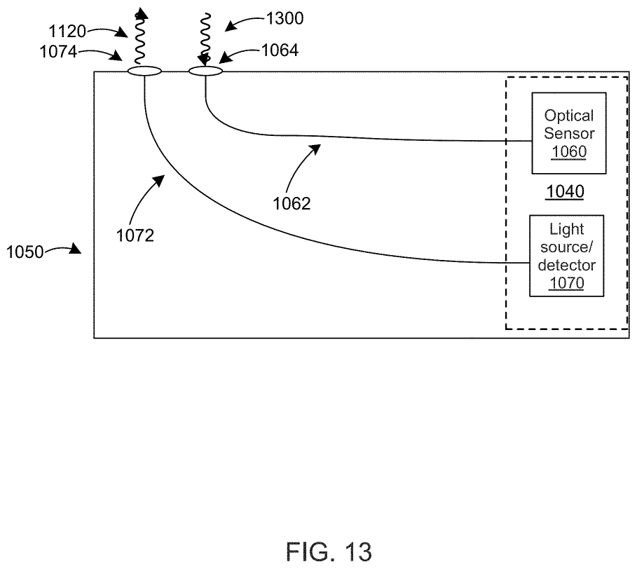

FIGS. 11 and 12 also illustrate the handle 1105 blocks all or substantially all light from entering optical port 1064, resulting in no light detection or a minimal light detection (e.g., below a threshold intensity) at optical sensor 1060. As discussed above, the detection of light intensity below a threshold intensity by optical sensor 1060 indicates that a therapeutic applicator (e.g., therapeutic applicator 1100) is present (i.e., mounted or disposed on therapeutic applicator support body 1050). When therapeutic applicator 1100 is not mounted or disposed on therapeutic applicator support body 1050, ambient light 1300 enters optical port 1064 as illustrated in FIG. 13. The ambient light 1300 is detected by optical sensor 1060 after passing through optical fiber 1062. As discussed above, the detection of light intensity at or above a threshold intensity by optical sensor 1060 indicates that a therapeutic applicator is not present (i.e., is not mounted or disposed on therapeutic applicator support body 1050).

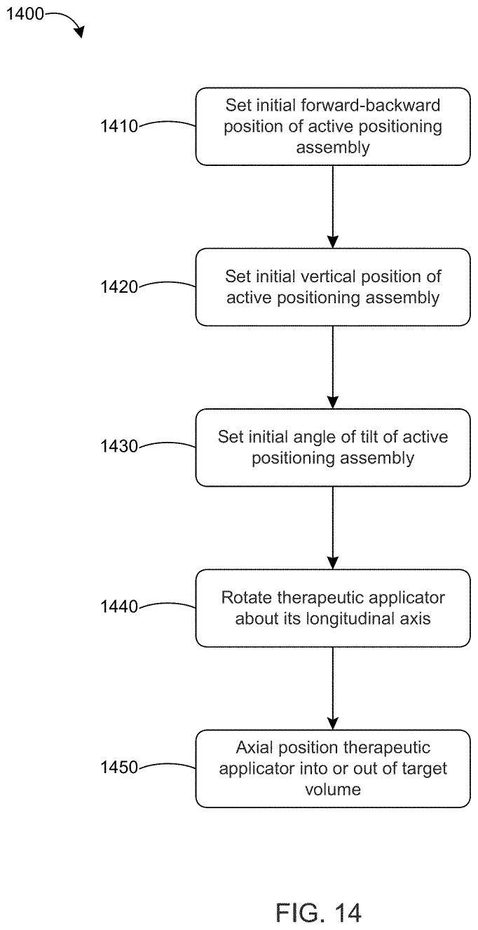

FIG. 14 is a flow chart 1400 of a method for positioning a therapeutic applicator with a positioning system that includes a passive positioning assembly and an active positioning assembly according to one or more embodiments. In step 1410, the initial forward-backward position of the active positioning assembly with respect to a base is set manually by horizontally sliding the passive positioning assembly with respect to the base. In step 1420, the initial vertical position of the active positioning assembly with respect to the base is set manually by vertically sliding the passive positioning assembly with respect to the base. In step 1430, the initial angle of tilt of the active positioning assembly with respect to the base is set manually by rotating the passive positioning assembly with respect to the base. In step 1440, the active positioning assembly rotates, electromechanically, the therapeutic applicator about a longitudinal axis of the therapeutic applicator, which is defined or determined by the initial angle of tilt of the active positioning assembly (e.g., because the therapeutic applicator is disposed on the active positioning assembly including a therapeutic applicator support body (e.g., therapeutic applicator support body 1050)). In step 1450, the active positioning assembly axially positions, electromechanically, the therapeutic applicator into or out of a target volume along the longitudinal axis of the therapeutic applicator.

In some embodiments, step 1440 can be performed by rotating a first motor (e.g., motor 1000) that is mechanical communication with a gear on the therapeutic applicator (e.g., via a lead screw such as first lead screw 1020). The rotational position of the therapeutic applicator can be determined by a rotation-position command received from a processor-based controller (e.g., via a motor controller interface).

In some embodiments, step 1450 can be performed by rotating a second motor that is mechanical communication with a distal portion of a therapeutic applicator support body to push or pull the distal portion with respect to a proximal portion of the therapeutic applicator support body. The axial position of the therapeutic applicator, and the distal portion of the therapeutic applicator support body, can be determined by an axial-position command received from a processor-based controller (e.g., via a motor controller interface).

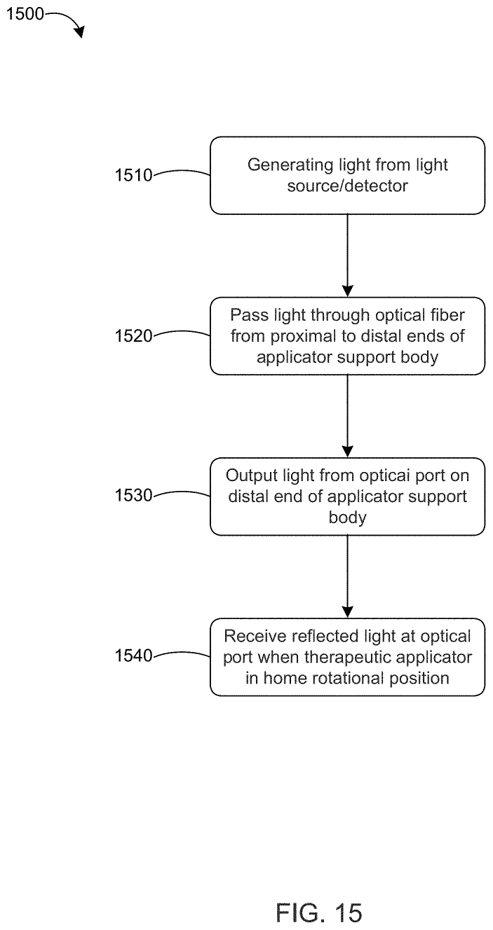

FIG. 15 is a flow chart 1500 of a method for indexing a rotational position of a therapeutic applicator according to one or more embodiments. In step 1510, light is generated from a light source/detector disposed in a proximal end of a therapeutic applicator support body. In some embodiments, the light source/detector is disposed in an EMI shielding chamber within the therapeutic applicator support body. In step 1520, the light passes through an optical fiber that is optically coupled to the light source/detector. In step 1530, the light is output from an optical port at a distal end of the therapeutic applicator support body. In step 1540, reflected light is received at the optical port when the therapeutic applicator is in a home rotational position. For example, the reflected light can be reflected by a reflective rotational indexing body attached or coupled to the therapeutic applicator.

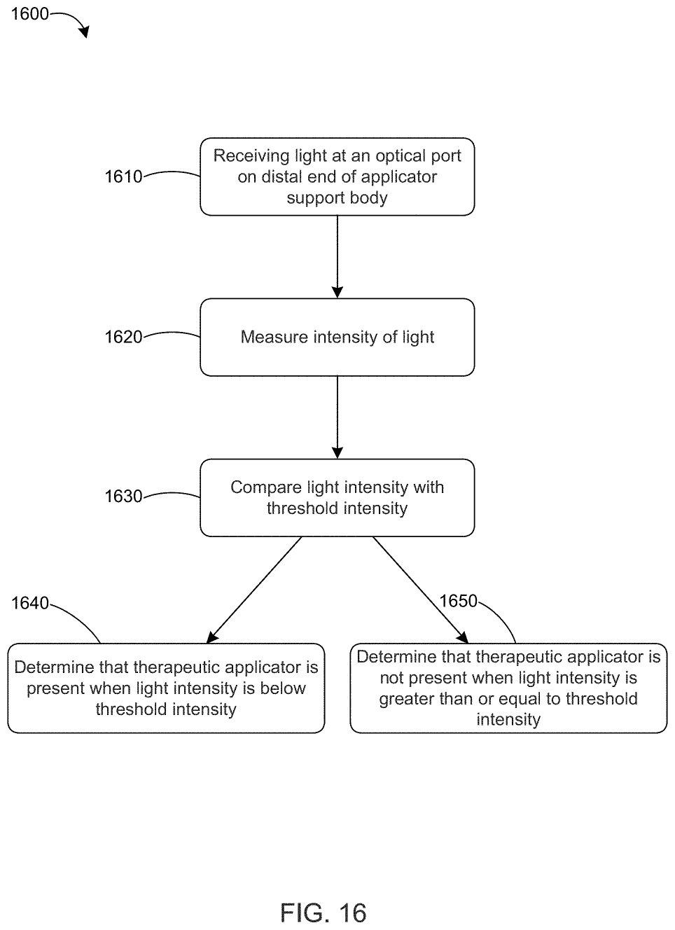

FIG. 16 is a flow chart 1600 of a method for determining a presence of a therapeutic applicator on a therapeutic applicator support body according to one or more embodiments. In step 1610, light is received at (or passes into) an optical port disposed on a distal end of the therapeutic applicator support body. The optical port is optically aligned with the therapeutic applicator. In step 1620, the light intensity is measured with a light detector disposed in proximal end of a therapeutic applicator support body. The light detector and the optical port can be optically coupled to an optical fiber that extends therebetween. In some embodiments, the light detector is disposed in an EMI shielding chamber within the therapeutic applicator support body. In step 1630, the measured light intensity is compared with a threshold light intensity. If the measured light intensity is less than the threshold light intensity, it is determined that the therapeutic applicator is present in step 1640. The measured light intensity is less than the threshold light intensity because the therapeutic applicator blocks or substantially blocks light from entering the optical port. If the measured light intensity is greater than or equal to the threshold light intensity, it is determined that the therapeutic applicator is not present in step 1640. The measured light intensity is greater than or equal to the threshold light intensity because a therapeutic applicator is not present allowing ambient light to enter the optical port.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of the present application need not reside on a single computer or processor, but may be distributed in a modular fashion among a number of different computers or processors to implement various aspects of the present application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the present disclosure and claims include new and novel improvements to existing methods and technologies, which were not previously known nor implemented to achieve the useful results described above. Users of the present method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. An applicator positioning system for positioning a therapeutic applicator with respect to a target volume, comprising:
a passive positioning assembly operated by a plurality of manual adjustors to control a corresponding plurality of passive degrees of freedom of said passive positioning assembly; and
an active positioning assembly operated by a plurality of rotary electric motors to control a corresponding plurality of active degrees of freedom of said active positioning assembly;
a motor housing unit, containing said plurality of rotary electric motors;
wherein the passive positioning assembly is configured to support and set the active positioning assembly into an initial position with respect to a base of said system in said plurality of passive degrees of freedom, including:
a first passive degree of freedom defined by a first translational adjustment unit slidably positioning the active positioning assembly at an initial forward-backward position with respect to the base, then securable in said initial forward-backward position;
a second passive degree of freedom defined by a second translational adjustment unit slidably positioning the active positioning assembly at an initial vertical position with respect to the base, then securable in said initial vertical position; and
a third passive degree of freedom defined by a tilt adjustment unit angularly tilting said active positioning assembly at an initial elevation angle of tilt with respect to said base, then securable at said initial elevation angle; and
wherein the active positioning assembly is configured to support and set the therapeutic applicator in a plurality of active degrees of freedom using a respective plurality of rotary electric motors disposed in said motor housing unit, including:
a first active degree of freedom defined by an angle of rotation of said therapeutic applicator about a longitudinal axis thereof, the longitudinal axis defined by the initial elevation angle of the active positioning assembly, said first active degree of freedom controlled by a first rotary electric motor within said motor housing, the first rotary electric motor coupled to a rotating shaft that causes rotation of said therapeutic applicator about its longitudinal axis within the target volume; and
a second active degree of freedom defined by axially translating the therapeutic applicator along the longitudinal axis of the applicator, said second active degree of freedom controlled by a second rotary electric motor within said motor housing, the second rotary electric motor coupled to and driving a lead screw that rotates and moves said therapeutic applicator along the longitudinal axis of the applicator for insertion and retraction of the therapeutic applicator into and out of the target volume, respectively.

2. The system of claim 1, wherein the passive positioning assembly is mechanically operated.

3. The system of claim 1, wherein the base is configured and arranged to mechanically mate to a patient support table.

4. The system of claim 1, wherein the rotating shaft of the first rotary electric motor turns a gear that engages a corresponding gear on the therapeutic applicator to cause the rotation of the therapeutic applicator.

5. The system of claim 1, wherein the lead screw of the second rotary electric motor engages a threaded hollow body coupled to the therapeutic applicator to cause the axial insertion and retraction of the therapeutic applicator.

6. The system of claim 1, wherein the therapeutic applicator comprises a treatment end insertable into said target volume and an opposing end not insertable into the target volume, said electromagnetically-shielding motor housing being disposed at said opposing end thereof.

7. The system of claim 1, wherein the motor housing unit is electromagnetically-shielding and comprises an electromagnetic interference shielding chamber having a grounded conducting layer surrounding said motors so as to reduce radio frequency interactions between said motors and an environment in which said system is operated.

8. The system of claim 1, each of said rotary electric motors comprising an electrically driven motor receiving respective electrical power signals and is respectively controlled by a processor based motor controller circuit within said motor housing unit.

9. The system of claim 6, further comprising an optical sensor configured and arranged to detect a rotational state of said therapeutic applicator about its longitudinal axis.

10. The system of claim 9, said optical sensor coupled to one or more fiber optic conduits carrying an optical signal to and/or from said optical sensor.

11. The system of claim 9, said optical sensor comprising a LED emitter and receiver configured and arranged to emit light towards an optical marker in said applicator and to receive a portion of said emitted light from said optical marker.

12. The system of claim 9, said optical sensor configured and arranged to detect a reference home position of said applicator.

13. An applicator positioning system for positioning a treatment applicator with respect to a patient support platform, the positioning system comprising:
a first positioning assembly mountable to said support platform;
a second positioning assembly mountable to the first positioning assembly;
wherein the first positioning assembly is equipped with:
a superior-inferior translational stage that positions the system at an initial horizontal position along an axis running in a superior-inferior direction with respect to said support platform;
an anterior-posterior translational stage that positions the system at an initial vertical position along an axis running in an anterior-posterior direction above said support platform; and
an elevation angle tilt stage that provides an initial elevation angle of tilt to said second positioning assembly and said applicator; and
wherein the second positioning assembly is equipped with:
a motorized and computer-controlled translational stage that positions the applicator along an axial location defined by an axis of said applicator so as to provide control of a linear insertion-and-retraction movement of said applicator along its axis, said motorized translational stage including a first rotary electric motor coupled to a lead screw rotating therewith to affect said insertion and retraction; and
a motorized and computer-controlled rotational stage that positions the applicator at an angle of rotation about its axis so as to provide rotational control of said applicator, said motorized rotational stage including a second rotary electric motor coupled to a shaft rotating therewith to affect said angular control.

14. The system of claim 13, further comprising an electrically-powered therapy treatment transducer apparatus proximal to a superior end of said applicator.

15. The system of claim 14, wherein said first positioning assembly is configured and arranged to provide any of said initial positionings prior to powering on the thermal therapy treatment transducer assembly.

16. The system of claim 14, wherein said second positioning assembly is configured and arranged to provide any of said linear and rotational motorized positionings during powering of the thermal therapy treatment transducer assembly.

17. The system of claim 13, said rotary electric motors disposed in an electromagnetically-shielding motor housing disposed distally from said superior end of the applicator.
